(19) United States
(12) Patent Application Publication
(10) Pub. No.: US 2003/0186401 A1
(43) Pub. Date: Oct. 2, 2003

(54) Method of recovering pinitol or chiro-inositol in high yield from soy fractions

(76) Inventors: Yong-Chul Shin, Jinju (KR); Yeong-Joong Jeon, Seoul (KR); Jong-Jin Kim, Jinju Gyeongsangnam-do (KR); Chi-Man Choi, Jinju Gyeongsangnam-do (KR)

Correspondence Address: David A Einhorn, Anderson Kill & Olick, 1251 Avenue of the Americas, New York, NY, 10020, US

(21) Appl. No.: 10/297,043

(22) PCT Filed: Nov. 20, 2001
(86) PCT No.: PCT/KR01/01984

(51) Int. Cl. C12P 7/02; C12P 19/02

(52) U.S. Cl. 435/155; 435/105

(57) ABSTRACT

This invention relates to a method for recovering useful products from soy fractions with high efficiency, and more specifically to increasing the recovery yield of pinitol or chiro-inositol from soy fractions, in which it is contained by a process comprising the steps of culturing a microorganism to transform pinitol derivatives into pinitol in soy fractions, thereby to increase the pinitol content in soy fractions, followed by removing microorganisms, insoluble materials and other macromolecules from said fractions by centrifugation or filtration to obtain an aqueous solution containing pinitol or chiro-inositol, contacting said solution with activated carbon to adsorb the pinitol or chiro-inositol, contacting said solution with activated carbon to adsorb the pinitol or chiro-inositol, and then recovering it by stepwise or gradient elution with an organic solvent.

### TECHNICAL FIELD

The present invention relates, in general, to the recovery of pinitol or chiro-inositol from soy fractions and, particularly, to a method of isolating pinitol or chiro-inositol at high efficiency with economic benefit from soybean curd whey, which is wasted after the production of soybean curd from soybean; or from soy molasses left after production of soy proteins; or from hydrothermal solution of defatted soybean meals, by microbial treatment and activated charcoal column chromatography.

### BACKGROUND ART

With recent increasing interest in health, various attempts have been made to develop novel health food materials. Considered as a health food material, soybean is now studied for its novel functions in the body.

In addition to being highly valuable in a sitological aspect, soybean is known to have a variety of physiological activities, including activities against cancer, arteriosclerosis, oxidation and bacteria, and blood glucose reduction. Responsible for such physiological activities, ingredients of soybean are exemplified by isoflavone, saponin, lecithin, trypsin inhibitor, etc. Isoflavone, which was proven effective in preventing cancer and osteoporosis, has been commercialized as a material for health foods in many advanced countries. Also, soybean oligosaccharides, such as raffinose and stachyose, were identified as being effective for promoting growth of beneficial intestinal bacteria, and commercialized in Japan.

In expectation of the existence of other biologically active materials in soybean, much active research has been conducted. As a result, soybean was found to contain chiro-inositol and its methyl ether derivative, called pinitol. Recently, these sugars have attracted particular attention since finding that they are useful in reducing serum glucose in type 2 (insulin-independent) diabetics.

As well known, chiro-inositol is an epimer of myo-inositol, and pinitol has a methyl group linked to the carbon at position 3 via an ether bond.

Since the early 1990s, the serum glucose-reducing effect of chiro-inositol has been verified in many reports (Ortmeyer et al., Endocrinol. 651, 1993; Huang et al., Endocrinol. 132:652-657, 1993; Farese et al., Proc. Natl. Acad. Sci. USA, 91:11040-11044, 1994; Fonteles et al., Diabetologia 39;731-734,1996). This naturally occurring compound is found to show no side effects, such as gastroenteric or hepatic troubles that conventional oral hypoglycemics have, and does not cause hypoglycemia even upon overuse. With this safety advantage, chiro-inositol has high possibility to be successfully developed as health food materials or medicines. Besides, chiro-inositol is suggested as being therapeutically effective for the treatment of obesity and polycystic ovarian syndrome (Nestler J. E. et al., New Eng. J. Med., 340:1314-1320, 1999). Pinitol, which is predominant over other chiro-inocitol derivatives in soybean, is also found to show the same hypoglycemic effect as that of chiro-inositol (U.S. Pat. No. 5,827,896; Narayanan et al., Current Science, 56(3):139-141, 1987).

Diverse methods have been suggested for preparing chiro-inositol, thus far. For example, hydrolysis of pinitol (methyl ether of D-chiro-inositol) extracted from plant leaves (Anderson et al., Ind. Eng. Chem., 45:593-596, 1953), and organochemical conversion of myo-inositol to chiro-inositol (Shen et al., Tetrahedron Letters, 131:1105-1108, 1990) are reported. However, these prior art methods are economically unfavorable because they take a long time and show low yield in the preparation of chiro-inositol. In addition, chiro-inositol can be synthesized from kasugamycin (U.S. Pat. No. 5,091,596). However, this method is low in efficiency, so that chiro-inositol is produced at high cost.

Aiming to more efficient production of chiro-inositol, the present inventors have researched the use of edible resources rich in chiro-inositol, such as soybean, its processed foodstuffs, and pine needles, as materials for reducing serum glucose levels (Korean Pat. Appl'n No. 10-2000-12881) and isolation and purification of chiro-inositol from such edible resources by acid hydrolysis (Korean Pat. Appl'n No. 10-2000-12882). Also, the present inventors developed the use of activated charcoal column in separating pinitol and chiro-inositol (Korean Pat. Appl'n No. 10-2001-001611), which is economically favorable in comparison with prior art methods, such as use of zeolite (U.S. Pat. No. 4,482,761), cation exchange resins (U.S. Pat. No. 5,096,594) and anion exchange resins (U.S. Pat. No. 5,482,631). In addition, the present inventors made a study of effective recovery of pinitol and chiro-inositol (Korean Pat. Appl'n No. 10-2001-44677), in which microorganisms such as bacteria, yeasts, and fungi are used to convert precursors existing as glycosides or phosphorus compounds to pinitol and chiro-inositol.

### DISCLOSURE OF THE INVENTION

Leading to the present invention, the intensive and thorough research into the economic production of pinitol or chiro-inositol(hereinafter both referred generally to as “chiro-inositol ingredients”), conducted by the present inventors, resulted in the finding that soy fractions contain chiro-inositol ingredients and are enriched in chiro-inositol ingredient content by treatment with certain microbes, and chromatography eluting with an organic solvent such as ethanol through a column packed with activated charcoal allows the chiro-inositol ingredients to be isolated with a purity of 90% or higher.

Therefore, it is an object of the present invention to provide a method for recovering chiro-inositol ingredients from soy fractions at high efficiency.

It is another object of the present invention to provide a recovery method of chiro-inositol ingredients, which is economically favorable.

It is a further object of the present invention to provide a method for recovering chiro-inositol ingredients and other useful ingredients from soy fractions, with ease.

In accordance with an aspect of the present invention, there is provided a method for recovering chiro-inositol ingredients from soy fractions, in which the soy fractions are provided in a liquid phase and the microbes are cultured in the soy fractions to increase the content of pinitol or chiro-inositol, said microbes being selected from the group consisting of bacteria, yeasts, fungi, or combinations thereof.

In accordance with another aspect of the present invention, there is provided a method for isolating chiro-inositol ingredients from soy fractions, comprising the steps of: providing the soy fractions as a liquid phase sample; removing insoluble matters and macromolecules from the liquid phase sample by centrifugation or filtration; passing the liquid phase sample removed of insoluble matters and macromolecules through a column packed with activated charcoal to adsorb the chiro-inositol ingredients onto the activated charcoal; washing the column with distilled water to remove molecules remaining unadsorbed; and eluting the adsorbate chiro-inositol ingredients with a stepwise or continuous concentration gradient of an aqueous organic solution, said aqueous organic solution ranging in concentration from 5 to 20% (v/v) and being selected from the group consisting of solutions of methanol, ethanol, isopropanol, and acetone in water.

In accordance with a further aspect of the present invention, there is provided a method for isolating chiro-inositol ingredients from soy fractions, comprising the steps of: providing the soy fractions as a liquid phase sample; culturing at least one microbial species in the liquid phase sample to increase the content of pinitol or chiro-inositol therein; removing the microbial mass generated during culturing, insoluble matters and macromolecules from the culture by centrifugation or filtration; and recovering pinitol or chiro-inositol from the supernatant or filtrate by activated charcoal column chromatography or ion exchange chromatography.

In accordance with still a further aspect of the present invention, there is provided a method for isolating chiro-inositol ingredients from soy fractions, comprising the steps of: providing the soy fractions in a liquid phase and concentrating them; culturing at least one microbial species in the concentrate to increase the content of pinitol or chiro-inositol therein, said microbial species being selected from the group consisting of bacteria, yeasts, and fungi; concentrating the culture to a solid content of 50-70% (w/w) and adding the concentrate with a 95% ethanol solution in an amount as large as one to three volumes of the remaining liquid portion of the concentrate to further precipitate insoluble matters; removing the solid content by centrifugation or filtration; vaporizing the ethanol contained in the supernatant or filtrate, said supernatant or filtrate being enriched in pinitol; passing the supernatant or filtrate deprived of ethanol through an activated charcoal column to adsorb pinitol or chiro-inositol onto the activated charcoal and eluting the adsorbate with an eluent; and concentrating the eluate and crystallizing chiro-inositol or pinitol.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a process flow illustrating the isolation of pinitol and chiro-inositol from soy fractions by use of an activated charcoal column;

FIG. 2 is a chromatogram obtained by column chromatography eluting pinitol (PI), chiro-inositol (CI) and oligosaccharides (OS) from a column of a size of 500 ml with 500 ml of 10% (v/v) ethanol and 500 ml of 50% (v/v) ethanol at a rate of 500 m/min;

FIG. 3 is a chromatogram obtained by column chromatography eluting pinitol (PI) and oligosaccharides (OS) from a column of a size of 10 L with 10 L of 10% (v/v) ethanol and 10 L of 50% (v/v) ethanol at a rate of 10 L/min;

FIG. 4 is a process flow associated with microbial treatment, illustrating the isolation of pinitol and chiro-inositol from soy fractions by use of an activated charcoal column; and

FIG. 5 is a process flow illustrating the production of pinitol of high purity according to a preferred embodiment of the present invention.

### BEST MODES FOR CARRYING OUT THE INVENTION

In general, the present invention is directed to the recovery of pinitol and/or chiro-inositol at high yield from natural resources, such as fruits of Glycine soja S. et Z., Glycine Max(L.) Merr., soybean, bean leaves, bean buds, defatted soybean, bean chaff, bean sprouts, pine needles, pine bud, inner layer of pine bark, etc.

In the present invention, microbes such as bacteria, yeasts, or fungi are cultured in edible resources containing pinitol or chiro-inositol, e.g., soy fractions, to increase the content of pinitol or chiro-inositol and these compounds are selectively adsorbed and eluted by use of a column packed with activated charcoal, whereby pinitol or chiro-inositol can be produced at high yield with economic benefit. Quite different from prior arts, the present invention can remove saccharides from soy fractions to bring about a significant reduction in the load of organics contained in the final waste, as well as recovering chiro-inositol ingredients in a small volume with a high concentration, thus isolating chiro-inositol ingredients at high efficiency.

The term “soy fractions” as used herein means soybean curd whey, which is generally wasted after the production of soybean curd, soy molasses left after the production of soybean proteins, a hydrothermal solution of defatted soybean meal, or mixtures thereof.

Generally, 1 g of soybean contains 3.4-6.8 mg of chiro-inositol ingredients that are found to exist as chiro-inositol at an amount of 15-20%, as pinitol at an amount of 25-35% and as chiro-inositol or pinitol glycosides or phosphorus compounds at an amount of 50-60%. Soybean curd whey also contains various chiro-inositol ingredients: 15-20% in the form of chiro-inositol; 30-40% in the form of pinitol; and the remainding 50-60% in the form of glycosides or phosphorous compounds of chiro-inositol or pinitol (in detail, pinitol glycosides, pinitol phosphates, pinitol phytates, pinitol phospholipids, pinitol esters, lipid-bound pinitol). Likewise, other soy fractions contain various chiro-inositol compounds in which glycosides or phosphorous compounds of pinitol exist in significant amounts, in spite of the predominance of pinitol itself. Different from pinitol in physical properties, such pinitol derivatives cannot be recovered by conventional pinitol recovery processes.

Leading to the present invention, the intensive and thorough research into the recovery of pinitol or chiro-inositol, conducted by the present inventors, resulted in the finding that, as time goes by after the generation of soybean curd whey, the chiro-inositol ingredient composition of the soybean curd whey shifts toward increased free chiro-inositol and pinitol, with no modulation in the total amount of chiro-inositol ingredients in the soybean curd whey. It was also found that the conversion of derivatives of chiro-inositol and pinitol into free chiro-inositol and pinitol, useful for reducing serum glucose levels, is owed to the biological action of the microbes living in the soybean whey, such as bacteria, yeasts, fungi, etc. In the present invention, pinitol derivatives contained in soy fractions can be converted into free pinitol through treatment with such microbes, thereby greatly improving the recovery of pinitol from soy fractions. In addition, other saccharides can be removed from the soy fractions, thereby brining about a significant reduction in the burden of subsequent recovery processes as well as in the load of organics contained in the final waste.

If they convert pinitol derivatives into free pinitol and are harmless, bacteria, yeasts and fungi all can be used in the present invention. Of course, the kind of the microbe used determines conversion conditions, recovery efficiencies of pinitol, and removal rates of other saccharides. Among the microbes, Sacharomyces calsbergensis is of special interest for the present inventors because it is found to have the highest ability to produce pinitol as well as to remove sugars. After being used to increase the content of chiro-inositol ingredients, microbes are removed from, for example, soybean curd whey and recovered in various subsequent processes.

In the present invention, dominant species were selected from among the microbes that naturally live in soybean curd whey and increase the pinitol content, and measured for pinitol production ability. The results are given in Table 1, below. To this end, soybean curd whey was autoclaved at 121° C. for 15 min and used as a medium in which two naturally separated yeast species and three bacterial species were cultured, alone or in combination, for 5 days with observation of the change in pinitol and chiro-inositol content.

TABLE 1
Pinitol Production Ability of Microbes Separated from Soybean
Curd Whey

| | Strain | Pinitol (mg/L) | Chiro-Inositol (mg/L) |
|---|---|---|---|
| Intact Whey | | 467 | 65 |
| Mixed Microbes | | 616 | 129 |
| Yeast | Y1 | 614 | 156 |
| | Y2 | 619 | 166 |
| Bacteria | B1 | 525 | 128 |
| | B2 | 625 | 142 |
| | B3 | 608 | 136 |

As apparent from the data of Table 1, all of the microbes, irrespective of their kinds, make a contribution to the increase of pinitol and chiro-inositol content. The same experiments performed with other microbes led to the conclusion that the increase of the pinitol content in soybean curd whey is made by all microbes rather than specific microbes. However, pinitol increase rates differ from one microbe species to another. In the assimilation ability of the saccharides contained in soybean curd whey, there is also difference among microbe species. To complete the present invention, there were selected microbes that satisfy the following conditions: 1) rapid pinitol production, 2) efficient removal of saccharides from whey, and 3) safety for food processing. The results are given in Table 2, below.

TABLE 2
Pinitol Production of Various Microbes from Soybean Curd Whey

| | | Pinitol | | |
|---|---|---|---|---|
| | | Production | Sugar | |
| | Strain | Rate | assimilation | Use¹ |
| Yeast | S. carlsbergensis | +++++ | +++++ | Beer |
| | S. cerevisae | ++++ | ++ | invertase |
| | S. pastorianus | +++ | ++ | Beer |
| | C. utilities | +++ | + | 5-adenylic |
| | | | | acid |
| Fungi | A. niger | ++++ | +++++ | α-amylase |
| | P. funiculosum | ++ | ++ | pectinase |
| | T. viride | ++++ | + | xylanase |
| Bacteria | B. stearothermophillus | +++ | + | α-amylase |
| | E. coli | ++++ | + | — |
| | P. amyloderamosa | ++ | + | isoamylase |

¹according to Japanese Food Additive Regulations

As seen in Table 2, the microbes having superb pinitol production rates can be exemplified by Saccharomyces calsbergensis, Saccharomyces cerevisiae, Aspergillus niger, Trichoderma viride, and E. coli with preference for Saccaromyces calsbergensis. In saccharide assimilation ability, Saccharomyces calsbergensis and Aspergillus niger are superior to the other microbes. Meanwhile, fungi including Aspergillus niger were observed to increase the pinitol content to day 2 or 3 of culturing, but decrease it after that. It is believed that fungi utilize the produced pinitol as a nutrient in the late culture stage owing to the shortage of other nutrients. The microbes summarized in Table 2 are regarded as safe for food processing because they acquired permission for at least one use according to Japanese Food Additive Regulations. Taken together, the data obtained in the experiments demonstrate that Saccharomyces calsbergensis is the most preferable for producing pinitol.

Pinitol production was also conducted using defatted soybean meal. In this regard, defatted soybean meal is powdered and dissolved in hot water and the powder remaining unsolved was removed. Then, the hydrothermal solution was treated with Saccharomyces calsbergensis. The final pinitol content was measured to be increased by 1.5-2.0 fold, compared to the initial content, as with soybean curd whey. In the case that the microbe was inoculated in the hydrothermal solution which was not removed of the undissolved defatted soybean meal powder, the final pinitol content was increased to 2.0-2.5 times the initial content. This is believed to be attributed to the fact that glycosides present in the powder are extracted to the hydrothermal solution during culturing.

An experiment was executed to determine whether the concentration increase of pinitol is attributed to certain enzymes produced from microbes. Saccharomyces calsbergensis and Aspergillus niger were cultured in soybean curd whey and filtered off through a 0.45μ filter. The soybean curd whey thus free of microorganisms was combined with another sterile soybean curd whey and incubated for two days. No increase was observed in pinitol concentration, nor the composition changes. From this fact, it can be inferred that the concentration increase of pinitol by microbes results from complex biological mechanisms of microbes, rather than actions of one or two enzymes. For use in inoculation with microbes, the soybean curd whey or defatted soybean meal may be concentrated by a factor of 10-20 fold.

In accordance with the present invention, the recovery of chiro-inositol ingredients from soybean curd whey, soy molasses, or hydrothermal extracts of defatted soybean meal resorts to the use of activated charcoal columns. Soybean curd whey or hydrothermal extracts of defatted soybean meals contain large quantities of oligosaccharides as well as pinitol and chiro-inositol. In the present invention, pinitol and chiro-inositol are separated from other saccharides by use of an activated charcoal column. Pinitol and chiro-inositol are adsorbed, together with other saccharides, onto activated charcoal, but can be eluted separately from other saccharides with a concentration gradient of an organic eluent such as ethanol.

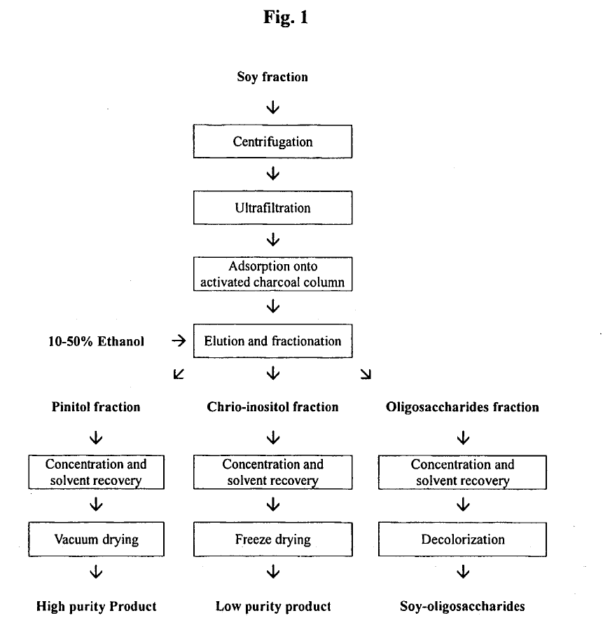

Below, a detailed description will be given of the recovery of chiro-inositol ingredients from soy fractions by use of an activated charcoal column in conjunction with FIG. 1.

In accordance with an aspect of the present invention, there is provided a method for recovering chiro-inositol ingredients from soy fraction using an activated charcoal column, which is broken down into the pretreatment process of removing insoluble ingredients and macromolecules such as proteins from soy fractions, the adsorption process of binding molecules of interest onto activated charcoal, the elution process of detaching the molecules from activated charcoal, and the post-treatment process of recovering chiro-inositol ingredients as powder. In the pretreatment process, insoluble matters or polymeric materials are removed by centrifugation or filtered off. The filtered sample is loaded onto a column filled with activated charcoal and chiro-inositol ingredients of the sample are adsorbed onto activated charcoal during passage through the column. After ingredients remaining unadsorbed are washed off with distilled water, the adsorbates are eluted with an organic solvent, such as methanol, ethanol, isopropanol or acetone. This eluent is fed at a continuous or stepwise concentration gradient from 5 to 20% (v/v). The activated charcoal is recycled by washing with distilled water. The eluate containing chiro-inositol is concentrated to a desired purity. More details are described as follows.

First Process: Pretreatment

In this pretreatment process, two tasks are performed in order to better the efficiency of the activated charcoal column process, a core process in the present invention. First, insoluble matters are removed from soy fractions with the aid of a centrifuge or filter. Insoluble matters, unless removed, clog the activated charcoal column to impede its normal absorption action. The other task is to remove macromolecules such as proteins from soy fractions by ultrafiltration. Once they are adsorbed onto activated charcoal, proteinaceous matters are hardly detached therefrom, thereby significantly decreasing the lifetime of the active column.

Second Process: Absorption into Activated Charcoal-Filled Column

In this process, a sample deprived of insoluble matters and proteinaceous matters in the pretreatment process is passed through an activated charcoal-filled column, during which chiro-inositol ingredients and soybean oligosaccharides are adsorbed onto the activated charcoal. To increase the absorption capacity of the activated charcoal, it is adjusted to the pH range of 6 to 8 with caustic soda. In one round, the column filled with the pH-controlled activated charcoal can take care of a soybean curd whey with a total solid content of 25 g chiro-inositol in an amount of as large as 5-10 volumes thereof. Preferably, the soybean curd whey is fed at a rate of 1-2 bed volumes (hereinbefore referred as to “BV”) per hour.

Third Process: Elution from Activated Charcoal Column

To remove matters that remain unadsorbed onto the activated charcoal, the column is washed with 1-2 BV of distilled water. Appropriate washing rates of the activated charcoal column fall into the range of 1-2 BV per hour. An eluent containing 5-20% (v/v) organic solvent is fed in an amount of 1-2 BV to elute from the activated charcoal chiro-inositol alone. Examples of the useful organic solvents include methanol, ethanol, isopropanol and acetone with highest preference for ethanol in consideration of workability, safety, and economic benefit. The eluent is preferably in the pH range of 3 to 4 at which chiro-inositol ingredients have low partition coefficients, and is fed at a rate of 0.5-2 BV per hour into the column. Afterwards, a regeneration solution containing the same solvent at a concentration of 40-80% is passed in an amount of 1 BV through the column to remove the matters adsorbed strongly onto the activated charcoal. Also, the regeneration solution is preferably fed at a rate of 0.5-1 BV per hour.

The eluate from the column is collected in fraction and analyzed by high performance liquid chromatography (HPLC), followed by pooling the fractions found to contain chiro-inositol ingredients. Those who have sufficient experience in this type of elution can harvest the fractions rich in chiro-inositol only by monitoring the changes of solvent concentration with the aid of a refractometer without additional fractionation work.

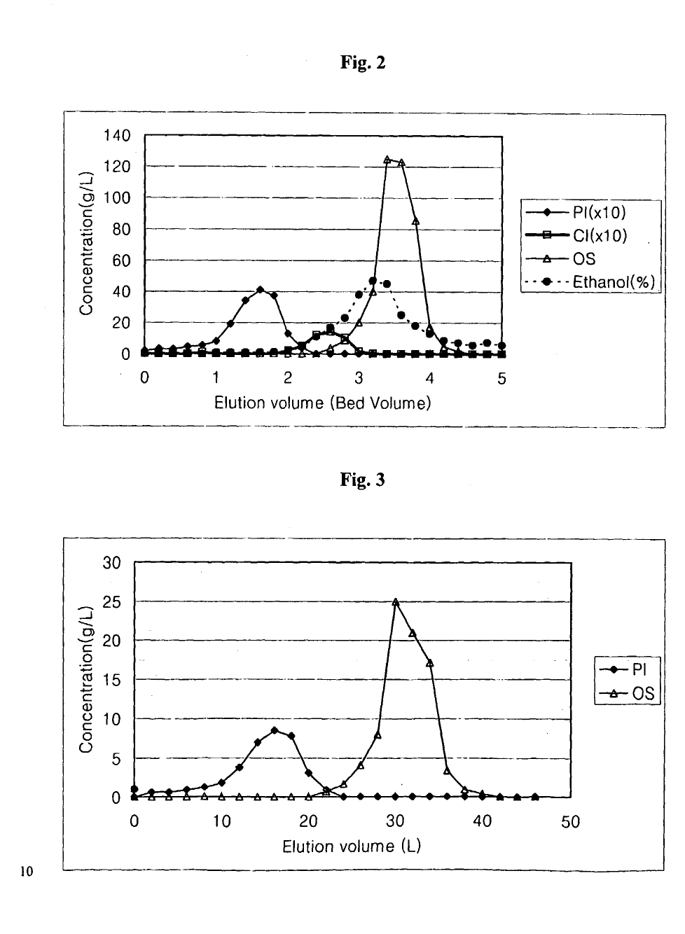

Under such eluting conditions, the adsorbates are eluted in the order of increasing adsorptive force. For example, pinitol fractions first come out of the column at 0.6-2.2 BV, then chiro-inositol fractions at 2.0-3.0 BV, and finally oligosaccharide fractions at 2.4-4.0 BV. In the pinitol fractions, the total content of chiro-inositol ingredients amounts to 55-70%, which is broken down into 50-60% of pinitol and 2-10% of chiro-inositol. The chiro-inositol fractions contain 5-10% of pinitol and 10-15% of chiro-inositol. Also, the chiro-inositol fractions further contain a significant amount of oligosaccharides, because the chiro-inositol peak is not completely separated from the oligosaccharide peak as shown in FIG. 2. The chiro-inositol ingredients contained in both the pinitol and the chiro-inositol fractions amount to 75-85% of the total chiro-inositol ingredients contained in the sample soy fraction. In the oligosaccharide fractions harvested at 3.0-4.0 BV, chiro-inositol ingredients are found at an amount of as low as 0.1-0.5% based on the total solid content.

After passage of the regeneration solution, 3-6 BV of distilled water is enough to remove the solvent remaining in the activated charcoal column, so that the washed activated charcoal column can be used for the next absorption.

Fourth Process: Concentration of Eluate and Recovery of Solvent

Vacuum distillation of each eluate fraction at 60° C. can achieve the concentration as well as the recovery of the solvent. The vacuum distillation is conducted to a solid content of 10% or higher. For use in the next round, the recovered solution containing the solvent is controlled in concentration.

Fifth Process: Drying

The concentrate obtained in the fourth process is freeze- or spray-dried to give products as white or yellow powder.

In accordance with the present invention, the above-mentioned processes can be modified, as follows, so as to isolate other useful ingredients from soy fractions or improve the production efficiency of chiro-inositol ingredients.

Modification 1: Associated with Recovery of Isoflavone

In advance of the second process, isoflavone can be recovered from the soy fractions. In this regard, the recovery of isoflavones resorts to adsorbents (HP resin, Samyang Corp. Korea) (Korean Pat. Laid-Open No. 2000-055133), or to alpha-galactosidase capable of cleaving isoflavone glycoside bonds (Korean Pat. Laid-Open No. 1998-032766). Where the adsorbents are used, pretreatment effects are also obtained because the adsorbents hold a significant amount of proteins thereto with no adsorption of chiro-inositol ingredients.

Modification 2: Associated with Recovery of Soybean Oligosaccharides

The oligosaccharide fractions obtained in the fourth process contain most of the oligosaccharides present in the soy fraction, as well as being highly free of impurities such as salts or proteins. Thus, soybean saccharide products of high quality can be produced only by simple purification processes.

Modification 3: Retreatment of Chiro-Inositol Fractions

By use of an additional activated charcoal column, the chiro-inositol fractions obtained in the fourth process can be purified to a higher chiro-inositol content. After being adjusted to the pH range of 3-4, the chiro-inositol fractions are subjected to activated charcoal column chromatography. At this pH, chiro-inositol is first eluted owing to its weak adsorptive force. The eluate fractions are pooled and can be processed to chiro-inositol products 50% or higher in purity. The retreatment may resort to the use of an anion exchange resin (U.S. Pat. No. 5,482,631).

Modification 4: Production of Pinitol Product with High Purity

The concentrate of the pinitol fractions obtained in the fourth process is further concentrated to a solid content of 50% or more and added with an equal volume of acetone at a low temperature, after which the solution is allowed to stand for 12 hours or more at 10° C. or less to give a precipitate. This pinitol matter is recovered by centrifugation or vacuum filtration and dried in vacuo to a purity of 95%.

Over conventional ion exchange resin process, the activated charcoal process of the present invention has the following advantages: 1) voluminous samples of low concentrations, such as soybean curd whey, can be treated because chiro-inositol ingredients strongly adsorb to activated charcoal and hardly detach therefrom until they meet specific elution conditions; 2) desalination is not needed in the pretreatment because activated charcoal allows salts of the sample to pass without retention; and 3) under appropriate elution conditions, high contents of chiro-inositol ingredients can be obtained in such relatively small volumes that the burden of concentrating the eluate, imposed on subsequent processes, is lightened.

In order to obtain chiro-inositol with higher purity, the activated charcoal column chromatography of the present invention may be further associated with treatment with microbes prior to the pretreatment process of removing impurities from soy fractions. In this regard, for example, soybean curd whey is treated with microbes to give a chiro-inositol ingredient-enriched solution which is then deprived of the biomass by centrifugation or filtration, lo followed by the recovery of chiro-inositol or pinitol of high purity through adsorption to the activated charcoal and other appropriate processes.

Therefore, in accordance with another aspect of the present invention, there is provided a method for recovering chiroinisitol ingredients from soy fractions, which comprises a microbial treatment process of increasing the content of pinitol or chiro-inositol in soy fractions by use of microbes; a pretreatment process of removing the microbial mass generated in the microbial treatment process, insoluble ingredients, and macromolecules such as proteins from soy fractions by filtration or centrifugation, an adsorption process of binding matters of interest onto a support by use of activated charcoal column chromatography or ion-exchange chromatography, an elution process of detaching the matters of interest from the support, and a post-treatment process of recovering chiro-inositol ingredients as powder.

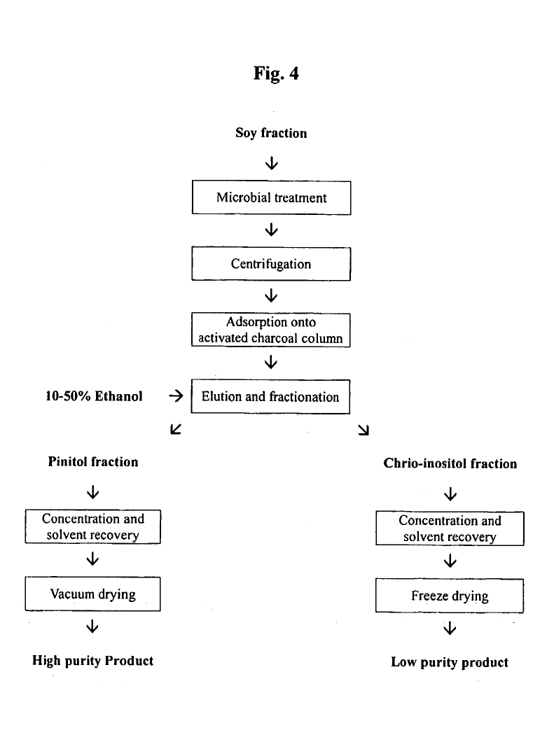

FIG. 4 shows a preferred embodiment of this method. First, soy fractions are concentrated and microbes such as bacteria, yeasts, or fungi are cultured in the concentrate to increase the content of pinitol or chiro-inositol. Centrifugation or filtration is conducted to remove the cultured microbes, insoluble matters and polymeric materials. The filtrate is loaded onto a column filled with activated charcoal and chiro-inositol ingredients of the filtrate are adsorbed onto activated charcoal during passage through the column. After ingredients remaining unadsorbed are washed off with distilled water, the adsorbates are eluted with ethanol. This eluent is fed at a continuous or stepwise concentration gradient from 10 to 50% (v/v). The activated charcoal is reused after washing with distilled water. The eluates containing chiro-inositol ingredients are concentrated to desired purity.

In order to produce pinitol with a purity of 90% or more, additional processes are conducted in addition to the basic processes of the method. A crystallization process is very useful and necessary for achieving a pinitol purity of as high as 90%. To allow the crystallization process, impurities which interrupt the crystallization of pinitol must be removed, in advance, to the extent that the solution prior to the crystallization has a pinitol purity of 70% or more. After the microbial treatment, the total solids of the soy fractions is analyzed to contain pinitol in an amount of as low as 5-7% with the remainder consisting of proteins, lipids, other carbohydrates, and salts. When this solution is applied to an activated charcoal column, other ingredients than pinitol give rise to a decrease in the capacity of the activated charcoal and the purity of the pinitol recovered is difficult to increase to higher than 70%. To avoid this problem, the solution after the microbial treatment is concentrated to an extent of a total solid content of 50-70% (w/w), after which the concentrate is added with one to three volumes of a 95% ethanol solution. In the resulting solution, all pinitol is dissolved in the supernatant while 75% or more of the other ingredients exist as precipitates. After removal of the precipitates by filtration or centrifugation, the supernatant is analyzed to have a pinitol content of 20% or more. Afterwards the ethanol added can be recovered in a significant amount by distillation and the remaining ethanol can be completely removed by a few rounds of distillation with addition of water. Then, the pinitol solution deprived of ethanol is loaded onto the activated charcoal column and the adsorbates are eluted with a 10% ethanol solution to give a pinitol purity of 70% or higher. As described above, the removal of impurities by solvent treatment prior to loading onto the activated charcoal column enjoys the advantage of producing pinitol in a high purity, increasing the capacity of the activated charcoal column, and preventing the lifetime of the activated charcoal from being reduced owing to the irreversible adsorption of proteins and other impurities. Subsequently, the eluate is concentrated to a pinitol concentration of 600 g chiro-inositol or more, followed by crystallization in ethanol to produce pinitol in a purity of 90% or more.

Therefore, in accordance with a further aspect of the present invention, there is provided a method for recovering pinitol in high purity from soy fractions, which comprises a microbial treatment process of increasing the content of pinitol or chiro-inositol in soy fractions by use of microbes after concentration of the soy fractions; a precipitation process of affording the microbial mass generated in the microbial treatment process, insoluble ingredients, and macromolecules such as proteins, as precipitates, a pretreatment process of removing the precipitates from soy fractions by filtration or centrifugation, an adsorption process of binding molecules of interest onto a support by use of activated charcoal column chromatography or ion-exchange chromatography, an elution process of detaching the molecules of interest from the support, and a post-treatment process of recovering chiro-inositol ingredients as powder.

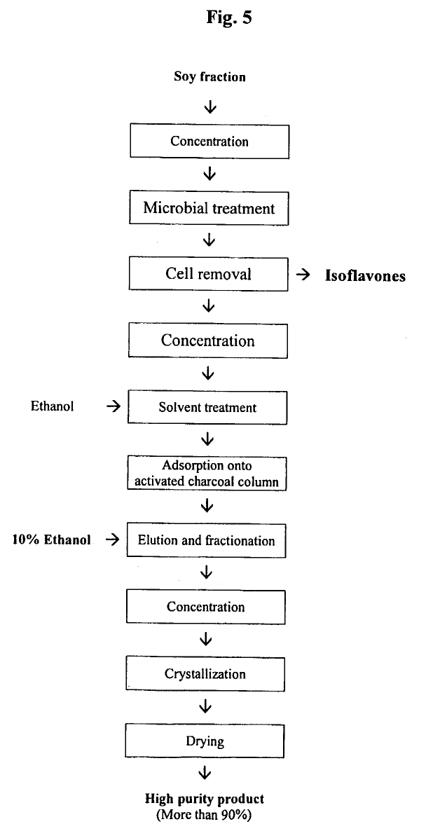

In FIG. 5, a preferred embodiment of this method is illustrated. First, soy fractions are concentrated and microbes such as bacteria, yeasts, or fungi are cultured in the concentrate to increase the content of pinitol or chiro-inositol. The chiro-inositol ingredient-enriched solution is concentrated to a total solid content of 50-70% (w/w) and added with one to three volumes of a 95% ethanol solution to give precipitates. Centrifugation or filtration is conducted to remove the precipitates. From the supernatant or filtrate, ethanol is removed by distillation. Subsequently, the solution free of ethanol is passed through an activated charcoal column to adsorb pinitol onto the activated charcoal, followed by elution with a 10% ethanol solution. The eluate is concentrated, and crystallization in ethanol gives pinitol with high purity.

When associated with other recovery processes, the method of the present invention can be used to isolate other useful ingredients, such as isoflavone and soybean oligosaccharides, from soy fractions, as illustrated above.

For use in drugs or foods for the treatment or prophylaxis of diabetes, obesity, or cataracts, the pinitol or chiro-inositol obtained in the present invention is formulated together with pharmaceutically acceptable carriers or added as a useful ingredient to functional beverages or foods.

A better understanding of the present invention may be obtained in light of the following examples which are set forth to illustrate, but are not to be construed to limit the present invention.

### EXAMPLE 1

### Treatment of Soybean Curd Whey with Saccharomyces calsbergensis

Saccharomyces calsbergensis was inoculated in sterile soybean curd whey and after 5 days of culturing, the soybean curd whey was analyzed for composition change. The results are given in Table 3, below. As seen in Table 3, the total pinitol was increased by a factor of 2.31 from 0.371 g/L to 0.857 g/L while the total sugar was reduced by 91%.

TABLE 3
Composition Change of Soybean Curd Whey after Treatment with
S. calsbergensis
(unit: g/L)

| | | | | | | | |
|---|---|---|---|---|---|---|---|
| | | | | | | C- | M- |
| Day | OD | T. solid | T. sugar | T. Protein | Pinitol | Inositol | Inositol |

| | | | | | | | |
|---|---|---|---|---|---|---|---|
| 0 | 2.00 | 30 | 12.9 | 6.62 | 0.371 | 0.134 | 0.039 |
| 1 | 9.08 | 20 | 6.1 | 6.42 | 0.528 | 0.083 | 0.083 |
| 2 | 11.81 | 18 | 2.5 | 6.42 | 0.590 | 0.080 | 0.022 |
| 3 | 12.80 | 15 | 1.3 | 6.64 | 0.648 | 0.070 | 0.005 |
| 4 | 12.73 | 14 | 1.2 | 6.55 | 0.722 | 0.080 | 0.004 |
| 5 | 12.53 | 14 | 1.2 | 6.75 | 0.857 | 0.088 | 0.004 |

### EXAMPLE 2

### Treatment of Soybean Curd Whey with Saccaromyces cerevisiae

Saccharomyces cerevisiae was inoculated in sterile soybean curd whey and after 5 days of culturing, the soybean curd whey was analyzed for composition change. The results are given in Table 4, below. As seen in Table 4, the total pinitol was increased by a factor of 1.81 from 0.371 g/L to 0.676 g/L while the total sugar was reduced by 54%.

TABLE 4
Composition Change of Soybean Curd Whey after Treatment with
S. cerevisiae
(unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 5.81 | 22 | 7.0 | 6.93 | 0.487 | 0.093 | 0.017 |
| 2 | 6.72 | 20 | 6.3 | 7.35 | 0.052 | 0.100 | 0.020 |
| 3 | 7.86 | 20 | 5.7 | 8.47 | 0.554 | 0.100 | 0.018 |
| 4 | 7.75 | 20 | 5.8 | 7.81 | 0.623 | 0.111 | 0.022 |
| 5 | 7.86 | 19 | 5.9 | 7.51 | 0.676 | 0.124 | 0.025 |

### EXAMPLE 3

### Treatment of Soybean Curd Whey with Saccaromyces pastorianus

Saccharomyces pastorianus was inoculated in sterile soybean curd whey and after 5 days of culturing, the soybean curd whey was analyzed for composition change. The results are given in Table 5, below. As seen in Table 5, the total pinitol was increased by a factor of 1.59 from 0.371 g/L to 0.590 g/L while the total sugar was reduced by 60%.

TABLE 5
Composition Change of Soybean Curd Whey after Treatment with
S. pastorianus
unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 9.20 | 21 | 7.1 | 7.19 | 0.495 | 0.090 | 0.015 |
| 2 | 10.78 | 20 | 6.2 | 6.45 | 0.535 | 0.110 | 0.017 |
| 3 | 10.68 | 20 | 4.6 | 7.12 | 0.566 | 0.122 | 0.022 |
| 4 | 10.57 | 20 | 5.7 | 7.07 | 0.595 | 0.120 | 0.023 |
| 5 | 10.53 | 19 | 5.2 | 6.96 | 0.590 | 0.123 | 0.023 |

### EXAMPLE 4

### Treatment of Soybean Curd Why with Candida utilitis

Candida utilitis was inoculated in sterile soybean curd whey and after 5 days of culturing, the soybean curd whey was analyzed for composition change. The results are given in Table 6, below. As seen in Table 6, the total pinitol was increased from 0.371 g/,L to the maximum 0.539 g/L after three days of culturing, and from then, its concentration decreased. The total sugar was reduced by as little as 31%.

TABLE 6
Composition Change of Soybean Curd Whey after Treatment with
C. utilitis
unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 11.01 | 21 | 7.3 | 7.27 | 0.483 | 0.105 | 0.019 |
| 2 | 11.05 | 20 | 6.3 | 7.83 | 0.523 | 0.123 | 0.023 |
| 3 | 11.12 | 20 | 6.2 | 9.37 | 0.539 | 0.124 | 0.025 |
| 4 | 11.01 | 20 | 6.6 | 8.67 | 0.477 | 0.130 | 0.007 |
| 5 | 10.82 | 20 | 8.9 | 8.43 | 0.407 | 0.118 | 0.008 |

### EXAMPLE 5

### Treatment of Soybean Curd Whey with Aspergillus niger

Aspergillus niger was inoculated in sterile soybean curd whey and after 5 days of culturing, the soybean curd whey was analyzed for composition change. The results are given in Table 7, below. As seen in Table 7, the total pinitol was increased from 0.371 g/L to the maximum 0.566 g/L after three days of culturing, and since then, decreased to almost zero after five days of culturing. The total sugar was reduced by as much as 94%.

TABLE 7
Composition Change of Soybean Curd Whey after Treatment with
A. niger
unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 0.35 | 28 | 13.3 | 6.12 | 0.406 | 0.102 | 0.056 |
| 2 | 0.15 | 20 | 5.7 | 2.81 | 0.472 | 0.155 | 0.023 |
| 3 | 1.30 | 10 | 0.7 | 1.04 | 0.566 | 0.176 | 0.005 |
| 4 | 1.24 | 8 | 1.3 | 1.16 | 0.230 | 0.052 | 0.000 |
| 5 | 1.18 | 8 | 0.8 | 1.28 | 0.060 | 0.000 | 0.000 |

### EXAMPLE 6

### Treatment of Soybean Curd Whey with Penicillium funiculosum

Penicillium funiculosum was inoculated in sterile soybean curd whey and after 5 days of culturing, the soybean curd whey was analyzed for composition change. The results are given in Table 8, below. As seen in Table 8, the total pinitol was increased from 0.371 g/L to the maximum 0.539 g/L after three days of culturing, and from then, its concentration decreased, while the total sugar was reduced by 49%.

TABLE 8
Composition Change of Soybean Curd Whey after Treatment with
P. funiculosum
unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 0.93 | 30 | 13.9 | 4.60 | 0.246 | 0.082 | 0.049 |
| 2 | 0.74 | 30 | 11.7 | 4.61 | 0.427 | 0.134 | 0.087 |
| 3 | 0.75 | 29 | 11.9 | 5.14 | 0.534 | 0.192 | 0.101 |
| 4 | 0.62 | 26 | 9.2 | 4.92 | 0.499 | 0.158 | 0.063 |
| 5 | 0.99 | 20 | 6.6 | 5.38 | 0.442 | 0.144 | 0.039 |

### EXAMPLE 7

### Treatment of Soybean Curd Whey with Trichoderma viride

Trichoderma viride was inoculated in sterile soybean curd whey and after 5 days of culturing, the soybean curd whey was analyzed for composition change. The results are given in Table 9, below. As seen in Table 9, the total pinitol was increased from 0.371 g/L to the maximum 0.709 g/L after one day of culturing, and since then, its concentration decreased, while the total sugar was reduced by 38%.

TABLE 9
Composition Change of Soybean Curd Whey after Treatment with
T. viride
unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 0.8 | 30 | 14.2 | 6.15 | 0.709 | 0.237 | 0.113 |
| 2 | 0.71 | 29 | 12.1 | 5.95 | 0.578 | 0.180 | 0.036 |
| 3 | 0.7 | 29 | 11.2 | 6.60 | 0.518 | 0.140 | 0.019 |
| 4 | 0.72 | 26 | 10.2 | 6.10 | 0.467 | 0.172 | 0.028 |
| 5 | 0.58 | 24 | 8.0 | 5.98 | 0.352 | 0.163 | 0.054 |

### EXAMPLE 8

### Treatment of Soybean Curd Whey with Bacillus stearothermophilus

Bacillus stearothermophilus was inoculated in sterile soybean curd whey and after 5 days of culturing, the soybean curd whey was analyzed for composition change. The results are given in Table 10, below. As seen in Table 10, the total pinitol was increased by a factor of 1.39 from 0.371 g/L to 0.514 g/L while the total sugar was reduced by 29%.

TABLE 10
Composition Change of Soybean Curd Whey after Treatment with
B. stearothermophilus
(unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 1.95 | 26 | 11.5 | 7.12 | 0.539 | 0.122 | 0.031 |
| 2 | 2.02 | 26 | 10.8 | 7.38 | 0.477 | 0.080 | 0.025 |
| 3 | 2.03 | 26 | 9.2 | 8.22 | 0.390 | 0.066 | 0.020 |
| 4 | 2.00 | 26 | 9.3 | 7.77 | 0.438 | 0.065 | 0.028 |
| 5 | 3.58 | 28 | 9.1 | 7.98 | 0.514 | 0.069 | 0.024 |

### EXAMPLE 9

### Treatment of Soybean Curd Whey with Escherichia coli

Escherichia coli was inoculated in sterile soybean curd whey and after 5 days of culturing, the soybean curd whey was analyzed for composition change. The results are given in Table 11, below. As seen in Table 11, the total pinitol was increased by a factor of 1.98 from 0.371 g/L to 0.733 g/L while the total sugar was reduced by 31%.

TABLE 11
Composition Change of Soybean Curd Whey after Treatment with
E. coli
(unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 3.6 | 26 | 11.4 | 7.79 | 0.613 | 0.079 | 0.043 |
| 2 | 3.59 | 24 | 8.8 | 7.44 | 0.643 | 0.075 | 0.045 |
| 3 | 3.50 | 25 | 8.7 | 8.47 | 0.660 | 0.070 | 0.047 |
| 4 | 3.35 | 24 | 8.4 | 7.62 | 0.712 | 0.082 | 0.063 |
| 5 | 3.41 | 24 | 8.9 | 8.43 | 0.733 | 0.085 | 0.072 |

### EXAMPLE 10

### Treatment of Soybean Curd Whey with Pesudomonas amylodermosa

Pseudomonas amylodermosa was inoculated in sterile soybean curd whey and after 5 days of culturing, the soybean curd whey was analyzed for composition change. The results are given in Table 11, below. As seen in Table 11, the total pinitol was increased from 0.371 g/L to the maximum 0.604 g/L after one day of culturing and since then, its concenration decreased, while the total sugar was reduced by as little as 24%.

TABLE 12
Composition Change of Soybean Curd Whey after Treatment with
P. amylodermosa
(unit: g/L)

| | | | | | | C- | M- |
|---|---|---|---|---|---|---|---|
| Day | OD | T. solid | T. sugar | T. Protein | Pinitol | Inositol | Inositol |
| 0 | 2.00 | 30 | 12.9 | 6.62 | 0.371 | 0.134 | 0.039 |
| 1 | 1.81 | 24 | 10.8 | 7.38 | 0.604 | 0.085 | 0.031 |
| 2 | 1.85 | 24 | 9.6 | 7.68 | 0.456 | 0.064 | 0.025 |
| 3 | 2.30 | 23 | 9.8 | 8.22 | 0.329 | 0.040 | 0.019 |
| 4 | 2.43 | 23 | 9.7 | 7.33 | 0.422 | 0.054 | 0.025 |
| 5 | 2.36 | 23 | 9.8 | 7.28 | 0.493 | 0.061 | 0.029 |

### EXAMPLE 11

### Treatment of Hydrothermal Solution and Suspension of Defatted Soybean with Saccharomyces calsbergensis

Saccharomyces calsbergensis was inoculated in a hydrothermal solution and a suspension of defatted soybean and cultured for five days. The media were analyzed for composition change during the culturing and the results are given in Table 13, below. After five days of culturing, as seen in Table 13, the pinitol content was increased by a factor of 2.02 from 0.596 g/L to 1.209 g/L in the hydrothermal solution of defatted soybean, and by a factor of 2.51 from 0.618 g/L to 1.551 g/L in the suspension of defatted soybean.

TABLE 13
Composition Changes of Hydrothermal Solution and Suspension of
Defatted Soybean after Treatment with S. calsbergensis

| | (unit: g/L) | | | | | | | |
|---|---|---|---|---|---|---|---|---|
| | Hydrothermal Extract | | | | Suspension | | | |
| Day | Pinitol | C-Inositol | T. solid | T. protein | Pinitol | C-Inositol | T. solid | T. Protein |
| 0 | 0.596 | 0.114 | 40 | 2.55 | 0.618 | 0.109 | 42 | 2.73 |
| 1 | 0.803 | 0.101 | 32 | 2.03 | 0.817 | 0.099 | 34 | 2.34 |
| 2 | 1.152 | 0.094 | 26 | 2.22 | 1.230 | 0.103 | 32 | 2.52 |
| 3 | 1.306 | 0.103 | 22 | 2.35 | 1.512 | 0.121 | 28 | 2.12 |
| 4 | 1.252 | 0.108 | 23 | 2.02 | 1.530 | 0.118 | 26 | 1.99 |
| 5 | 1.209 | 0.112 | 24 | 1.63 | 1.551 | 0.115 | 24 | 1.85 |

### EXAMPLE 12

### Isolation of Chiro-Inositol Ingredients Using Activated Charcoal Column

Experiment 1: Partition Coefficient for Adsorption Onto Activated Charcoal According to pH

To determine whether activated charcoal was suitable for isolating/L ingredients from other sugars, partition coefficients of chiro-inositol, pinitol and sugar for adsorption onto activated charcoal were examined at various pHs to pH. The results are given in Table 14, below. Herein, the term “adsorption partition coefficient” means the ratio of the concentration of an ingredient adsorbed onto an adsorbent to the concentration of the ingredient remaining unadsorbed under given conditions.

TABLE 14
Partition coefficients of Chiro-inositol, Pinitol and Sugar for
Adsorption onto Activated charcoal

| pH | Chiro-inositol | Pinitol | Sugar |
|---|---|---|---|
| 3 | 1.8 | 1.0 | 7.5 |
| 4 | 2.0 | 1.6 | 7.5 |
| 5 | 4.1 | 2.2 | 7.5 |
| 6 | 5.5 | 2.2 | 7.5 |
| 7 | 5.5 | 2.2 | 7.8 |
| 8 | 8.4 | 2.0 | 7.8 |
| 9 | 4.2 | 1.6 | 7.5 |

As indicated in Table 14, all chiro-inositol, pinitol and sugars have high partition constants for adsorption onto activated charcoal at around neutral pH. Thus, after adsorption is performed under such pH conditions, elution at acidic pH allows chiro-inositol and pinitol to come out ahead of sugars, thereby separating the two ingredients from sugars with ease.

Experiment 2: Composition of Soybean Curd Whey

The soybean curd whey used in the present invention was obtained from a soybean curd manufactory in Korea, and analyzed for composition. The results are given in Table 15, below.

TABLE 15
Composition of Soybean Curd Whey

| Ingredient | Content in Solid (%) | Content in Sol'n (g/L) |
|---|---|---|
| Chiro-inositol | 0.52 | 0.13 |
| Pinitol | 2.04 | 0.51 |
| Total Sugar | 45.0 | 11.25 |
| Protein | 12.0 | 3.00 |
| Ash | 3.0 | 0.75 |
| Others | 37.44 | 9.36 |
| Sum | 100.0 | 25.00 |

Experiment 3: Hydrothermal Extraction of Defatted Soybean Meal

200 g of the powder obtained by passing defatted soybean meal through a 20 mesh sieve was added with 800 ml of water and extracted at 80° C. for 2 hours with stirring. The hydrothermal solution was centrifuged at 10,000 rpm to give 600 ml of an aqueous defatted soybean solution (solid content 8.3% (w/v)). This extract was analyzed for composition and the results are given in Table 16, below.

TABLE 16
Composition of Hydrothermal Extract of Defatted Soybean Meal

| Ingredient | Content in Solid (%) | Content in Sol'n (g/L) |
|---|---|---|
| Chiro-inositol | 0.44 | 0.36 |
| Pinitol | 1.86 | 1.54 |
| Total Sugar | 39.0 | 32.4 |
| Protein | 18.0 | 14.9 |
| Ash | 4.0 | 3.3 |
| Others | 36.7 | 30.5 |
| Sum | 100.0 | 83.0 |

Experiment 4: Composition of Soy Molasses

Soy molasses obtained from various sources was analyzed for composition and the results are given in Table 17, below

TABLE 17
Composition of Soy Molasses

(unit g/100 g)

| Manufacturer | Aarhus | Central Soya | Solbar |
|---|---|---|---|
| (Nation) | (Dutch) | (U.S.A.) | (Israel) |
| Brix | 66.5 | 60 | 74 |
| Chiro-inositol | 0.4 | 0.3 | 0.6 |
| Pinitol | 1.5 | 1.0 | 1.7 |
| Total Sugar | 36.9 | 28.7 | 47.0 |
| Protein | 9.3 | 7.6 | 9.0 |
| Ash | 4.0 | 5.2 | 6.7 |

Experiment 5: Recovery of Chiro-Inositol Ingredients from Soybean Curd Whey

4 liters of soybean curd whey with the composition of Experiment 2 was filtered to remove insoluble solids, after which the filtrate was adjusted to pH 8.0 and loaded at a rate of 500 ml per hour onto a glass column (inner diameter 5 cm×length 30 cm) packed with 500 ml of activated charcoal. Used was granular activated charcoal with a size of 30-80 meshes. After completion of the adsorption of the filtrate onto activated charcoal, impurities remaining unadsorbed were removed by the passage of 500 ml of distilled water. Afterwards, flowing with 500 ml of a 10% (v/v) ethanol solution and then with a 50% (v/v) ethanol solution at a flow rate of 500 ml/hr eluted the adsorbates. Thereafter, passage of 2 liters of distilled water through the activated charcoal column made the ethanol remaining therein come out and it was reused in the next adsorption task. The effluent obtained over the elution period from the start of the elution to the completion of the washing was collected in fractions of 100 ml. Each fraction was analyzed for contents of pinitol, chiro-inositol and saccharides (sucrose, stachyose, raffinose, fructose and glucose) by HPLC using Dionex Carbonpak MA-1 (Dionex, U.S.A.) as an analysis column with the aid of a pulsed electrochemical detector, eluting with 69 mM NaOH at a rate of 0.4 ml/min for 90 min. Gas chromatography was used to analyze the concentration of ethanol in each fraction. In FIG. 2, contents of the compounds in each fraction are shown. As seen in FIG. 2, pinitol was eluated at 0.6-2.2 BV with a maximum concentration at 1.6 BV. Chiro-inositol was found mainly in the fractions in the range of 2.0 to 3.2 BV with a maximum concentration at 2.6 BV. As for oligosaccharides, they emerged when eluting at 2.4-4.2 BV and their concentration reached at 3.4 BV. The fractions in the range of 2.4-4.2 BV showing pinitol peaks were pooled to give 800 ml which was analyzed to contain pinitol in an amount of 2.03 g/L, chiro-inositol in an amount of 0.11 g/L, and oligosaccharides in an amount of 0.20 g/L. The content of the chiro-inositol ingredients amounted to 61.2% based on the total dry weight of the pinitol fractions. Likewise, the fractions in the range of 2.4-2.8 BV showing chiro-inositol peaks were pooled to give 300 ml which was analyzed to contain pinital in an amount of 0.1 g/L, chiro-inositol in an amount of 1.22 g/L, and oligosaccharides in an amount of 4.10 g/L. The content of the chiro-inositol ingredients in the chiro-inositol fraction pool was 16% based on the total dry weight of the pool.

Experiment 6: Recovery of Chiro-Inositol Ingredients from Hydrothermal Extract of Defatted Soybean Meal

1.5 liters of a hydrothermal extract of defatted hydrothermal meal having the composition of Experiment 3 was treated in the same manner as in Experiment 5 to give 900 ml of pinitol fractions and 300 ml of chiro-inositol fractions which contained chiro-inositol ingredients in amounts of 1.92 g/L and 1.10 g/L, respectively, based on the total solid weight.

Experiment 7: Recovery of Chiro-Inositol Ingredients from Soy Molasses

200 g of soy molasses having the composition shown in Table 17 of Experiment 4, produced in U.S.A., was diluted with distilled water to a volume of 1.5 liters. This dilution was treated in the same manner as in Experiment 5 to give 800 ml of pinitol fractions and 300 ml of chiro-inositol fractions which contained chiro-inositol ingredients in amounts of 1.88 g/L and 1.40 g/L, respectively, based on the total solid weight.

Experiment 8: Concentration and Drying of Eluate

800 ml of the pinitol pool obtained in Experiment 5 was concentrated to a volume of 40 ml in an evaporator maintained at 50° C. under vacuum. The concentrate was freeze-dried to give 3.0 g of a pale yellow powder which was analyzed to contain chiro-inositol ingredients in an amount of 63.1%. Likewise, 300 ml of the chiro-inositol pool was concentrated to a volume of 30 ml, followed by freeze-drying the concentrate to give 2.4 g of a pale yellow powder. In this powder, chiro-inositol ingredients were found to amount to 16.5%.

### EXAMPLE 13

### Recovery of Pinitol at High Efficiency

100 L of soybean curd whey with a pinitol content of 0.387 g/L was boiled for 20 min to kill autogenous microbes, cooled to 30° C., and inoculated with 2 L of precultured Saccharomyces calsbergensis. After incubation for 72 hours with ample supply of air, centrifugation was conducted to remove the cell mass and insoluble solid contents. The supernatant, amounting to 95 L, was measured to be increased to 0.793 g/L in pinitol content with no modulation in chiro-inositol content. The liquid was passed at a rate of 20 L/hour through a column packed with 10 L of activated charcoal to adsorb pinitol onto the activated charcoal. Following washing the activated charcoal with 10 L of distilled water, elution of pinitol was carried out with 10 L of 10% ethanol at a flow rate of 10 L/hr. Afterwards, 10 L of 50% ethanol was flowed at a rate of 10 L/hr into the activated charcoal column to remove saccharides therefrom. For use in the next adsorption, the activated charcoal was washed with distilled water.

From the start of eluent feeding, the eluates were collected in fraction by 2 L. Each fraction was analyzed for contents of pinitol and total saccharides and the results are given in FIG. 3. The fractions showing a pinitol peak were pooled to 14 L which was then freeze-dried to give 90.2 g of a pale yellow powder with a pinitol purity of 68.0%. Chiro-inositol fractions were not recovered owing to low chiro-inositol contents.

### EXAMPLE 14

### Production of Pinitol of High Purity

1,000 L of soybean curd whey with a pinitol concentration of 0.35 g/L was 10-fold concentrated to 100 L. The concentrate was cooled to 30° C. and inoculated with Saccharomyces calsbergensis in the same manner as in Example 13 in a 150 L tank. The yeast was incubated for 48 hours with sufficient aeration, followed by centrifugation to remove the cell mass and floating matters. The supernatant, amounting to 95.5 L, was analyzed to contain a pinitol content of 7.78 g/L, and 5-fold concentrated to 19.1 L. Adding 30 L of a 95% ethanol solution to the concentrate led to removal of 76.5% of the solid content present in the concentrate while the purity of pinitol reached 25% with a 4-fold increase. From the dilution, the ethanol was almost completely removed by repeating the addition and evaporation of water. 20 L of the ethanol-removed solution was loaded onto a 20 L activated charcoal column in the same manner as in Example 13 to adsorb pinitol onto the activated charcoal. Following washing with 20 L of distilled water, pinitol was eluted with 20 L of a 10% ethanol solution. The eluate was 20-fold concentrated to 1 L, added with 1.5 L of a 95% ethanol solution, and allowed to stand for 12 hours at room 5 temperature with slow stirring to give pinitol as a precipitate. It was recovered by vacuum filtration and dried at 40° C. in vacuo to give 495 g of a white powder with a pinitol purity of 96.5%. The material balance of the whole process is summarized in Table 18.

TABLE 18
Material Balance of the Process for High Purity Pinitol Production

| Process | Vol. (L) | Conc. (g/L) | T. Solid (g) | PI Content (g/L) | PI (g) | PI Purity (%) | Recovery Effic. (%) |
|---|---|---|---|---|---|---|---|
| Whey | 1000 | 18 | 18,000 | 0.35 | 350 | 1.9 | |
| Concentrated | 100 | 178 | 17,800 | 3.49 | 349 | 2.0 | |
| Whey | | | | | | | |
| Microbial | 95.5 | 126 | 12,033 | 7.78 | 743 | 6.2 | 100 |
| Treatment & | | | | | | | |
| Centrifugation | | | | | | | |
| Concentration | 19.1 | 630 | 12,020 | 38.9 | 742 | 6.2 | 99.9 |
| Solvent Add | 49.1 | 57.6 | 2,828 | 14.1 | 691 | 25.0 | 93.0 |
| Adsorption & | 20.0 | 45.0 | 832 | 31.3 | 626 | 75.2 | 84.3 |
| Elution | | | | | | | |
| Concentration | 2.5 | 359.6 | 832 | 250.0 | 625 | 75.1 | 84.1 |
| Crystallization | 495 g | 99.0% | 490 | 96.5% | 478 | 97.5% | 64.3 |
| & Drying | | | | | | | |

### INDUSTRIAL APPLICABILITY

As described hereinbefore, the method of the present invention can convert pinitol derivatives of soy fractions to pinitol by microbial treatment, thereby recovering pinitol at maximum efficiency. Additionally, other saccharides can be removed from the soy fractions, thereby brining about a significant reduction in the burden of subsequent recovery processes as well as in the load of organics contained in the final waste. Further, the activated charcoal column chromatography of the present invention enables the treatment of more voluminous samples of low concentrations, compared to ion exchange chromatography. Another advantage of the present invention is that desalination is not needed in the pretreatment because activated charcoal allows salts of the sample to pass without retention. The present invention also enjoys the advantage that, under appropriate elution conditions, high contents of chiro-inositol ingredients can be obtained in such relatively small volumes that the burden of concentrating the eluate, imposed on subsequent processes, is lightened. Consequently, the novel activated charcoal column chromatography associated with the microbial treatment can isolate chiro-inositol ingredients from soy fractions at far higher efficiency than can prior arts.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A method for recovering chiro-inositol ingredients from soy fractions, in which the soy fractions are provided in a liquid phase and the microbes are cultured in the soy fractions to increase the content of pinitol or chiro-inositol, said microbes being selected from the group consisting of bacteria, yeasts, fungi, or combinations thereof.

2. The method as set forth in claim 1, wherein said microbes belong to the genus Saccharomyces.

3. The method as set forth in claim 1, wherein said microbes belong to the genus Aspergillus.

4. The method as set forth in claim 2, wherein said microbes are Saccharomyces calsbergensis.

5. The method as set forth in claim 3, wherein said microbes are Aspergillus niger.

6. A method for isolating chiro-inositol ingredients from soy fractions, in which the soy fractions are provided in a liquid phase and passed through activated charcoal to adsorb the chiro-inositol ingredients and other saccharides onto the activated charcoal.

7. The method as set forth in claim 6, wherein the chiro-inositol ingredients comprise chiro-inositol and pinitol.

8. The method as set forth in claim 6, wherein the chiro-inositol ingredients are eluated with an aqueous solution of an organic solvent.

9. The method as set forth in claim 8, wherein the organic solvent is selected from the group consisting of ethanol, isopropanol, methanol and acetone.

10. The method as set forth in claim 8, wherein the chiro-inositol ingredients are eluted separately from other saccharides by increasing the concentration of the aqueous solution stepwise or continuously.

11. The method as set forth in claim 10, wherein the aqueous solution has a concentration of 1-20% in the early elution stage and 20-100% in the final elution stage.

12. A method for isolating chiro-inositol ingredients from soy fractions, comprising the steps of:
providing the soy fractions as a liquid phase sample; removing insoluble matters and macromolecules from the liquid phase sample by centrifugation or filtration;
passing the liquid phase sample removed of insoluble matters and macromolecules through a column packed with activated charcoal to adsorb the chiro-inositol ingredients onto the activated charcoal;
washing the column with distilled water to remove molecules remaining unadsorbed; and
eluting the adsorbate chiro-inositol ingredients with a stepwise or continuous concentration gradient of an aqueous organic solution, said aqueous organic solution ranging in concentration from 5 to 20% (v/v) and being selected from the group consisting of solutions of methanol, ethanol, isopropanol, and acetone in water.

13. A method for isolating chiro-inositol ingredients from soy fractions, comprising the steps of:
providing the soy fractions as a liquid phase sample;
culturing at least one microbial species in the liquid phase sample to increase the content of pinitol or chiro-inositol therein;
removing the microbial mass generated during culturing, insoluble matters and macromolecules from the culture by centrifugation or filtration; and
recovering pinitol or chiro-inositol from the supernatant or filtrate by activated charcoal column chromatography or ion exchange chromatography.

14. The method as set forth in claim 13, wherein the recovery step is carried out by the method claimed in any of claims 7 to 12.

15. The method as set forth in any one of claims 6 to 11, further comprising the step of recovering useful ingredients other than chiro-inositol ingredients from the soy fractions, said useful ingredients comprising isoflavone or soybean oligosaccharides.

16. A method for isolating chiro-inositol ingredients from soy fractions, comprising the steps of:
providing the soy fractions in a liquid phase and concentrating them;
culturing at least one microbial species in the concentrate to increase the content of pinitol or chiro-inositol therein, said microbial species being selected from the group consisting of bacteria, yeasts, and fungi;
concentrating the culture to a solid content of 50-70% (w/w) and adding the concentrate with a 95% ethanol solution in an amount as large as one to three volumes of the remaining liquid portion of the concentrate to further precipitate insoluble matters;
removing the solid content by centrifugation or filtration;
vaporizing the ethanol contained in the supernatant or filtrate, said supernatant or filterate being enriched in pinitol;
passing the supernatant or filtrate deprived of ethanol through an activated charcoal column to adsorb pinitol or chiro-inositol onto the activated charcoal and eluting the adsorbate with an eluent; and
concentrating the eluate and crystallizing chiro-inositol or pinitol.

17. The method as set forth in claim 1, 6 or 12, wherein the soy fractions are selected from the group belonging to the following categories:
(i) soybean, or defatted soybean meal or its extracts,
(ii) soybean curd whey
(iii) soy molasses
(iv) concentrates of (i) or (ii).

18. The method as set forth in claim 1, 6, 12, 13 or 16, wherein the chiro-inositol ingredients comprise chiro-inositol and pinitol.
(iii) soy molasses
(iv) concentrates of (i) or (ii).

19. The method as set forth in claim 1, 6, 12, 13 or 6, wherein the chiro-inositol ingredients comprise chiro-inositol and pinitol.
